(19) United States
(12) Patent Application Publication
(10) Pub. No.: US 2025/0207071 A1
(43) Pub. Date: Jun. 26, 2025

(54) SYSTEMS AND METHODS FOR CONTROLLING ATTENUATION IN A FERMENTATION PROCESS

(71) Applicant: Rhinegeist, LLC, Cincinnati, OH (US)

(72) Inventor: Nicholas Ketchum, Cincinnati, OH (US)

(21) Appl. No.: 19/073,982

(22) Filed: Mar. 7, 2025

Related U.S. Application Data
(63) Continuation of application No. 17/199,911, filed on Mar. 12, 2021, now Pat. No. 12,275,923.
(60) Provisional application No. 63/135,147, filed on Jan. 8, 2021.
(60) Provisional application No. 62/988,655, filed on Mar. 12, 2020.

(51) Int. Cl. C12C 11/00; C12C 5/00; C12H 1/00; C12Q 1/18; C12Q 1/6895

(52) U.S. Cl. CPC C12C 11/00; C12C 5/00; C12H 1/003; C12Q 1/18; C12Q 1/6895

(57) ABSTRACT

A method of controlling attenuation in a fermentation process includes culturing a fungus in a medium to produce a fermented liquid and adding a killer toxin to the fermented liquid. The killer toxin is added in an amount effective to deactivate the fungus.

### CROSS REFERENCE TO RELATED APPLICATIONS

This application is a continuation of U.S. patent application Ser. No. 17/199,911 filed on Mar. 12, 2021, which claims the priority benefit of U.S. Provisional Patent Application No. 63/135,147, filed Jan. 8, 2021, and U.S. Provisional Patent Application No. 62/988,655, filed Mar. 12, 2020, each of which is hereby incorporated herein by reference in their entirety.

### TECHNICAL FIELD

Embodiments of the technology relate, in general, to fermentation technology, and in particular to systems and methods for controlling attenuation in a fermentation process.

### BACKGROUND

In recent years, the negative effects of Saccharomyces cerevisiae var. diastaticus on the brewing community have been highlighted by increasing incidents of beer fermentations being infected with this pernicious beer spoilage yeast. Among different species that are capable of spoiling beer are a group of yeasts that are capable of expressing STA genes, these yeasts are often described as being diastatic. These genes, STA1, STA2, and STA3, allow yeasts to produce glucoamylase enzymes. These enzymes allow the yeast to break down dextrin left behind in the beer after the primary fermentation has consumed the saccharides created in the mashing process. The breakdown of dextrin into smaller mono/di/tri saccharides, which are subsequently fermented, is often referred to as over attenuation or hyper attenuation referring to the drop in the density of the beer as an indirect measurement of the concentration of sugars. Over-attenuation can produce gushing packages and off flavors in the finished product.

Currently, attempts at controlling diastatic yeasts infections are focused around screening and destruction of contaminated product or the wholesale pasteurization of product lines. However, many of the dominant beer styles that are produced in craft breweries are negatively affected by pasteurization. For example, beer styles that have been aggressively hopped post boil, such as India Pale Ales and Pale Ales, will suffer disproportionately from hop oxidation when pasteurized. Even when pasteurization is not prohibited by capital costs, many breweries will opt to not pasteurize their beer as it will cause degradation of the delicate hop aromas. A need exists for methods to stabilize beer brewed in the presence of diastatic yeasts that would otherwise have to be destroyed.

### SUMMARY

In an embodiment, a method of controlling attenuation in a fermentation process includes culturing a fungus in a medium to produce a fermented liquid and adding a killer toxin to the fermented liquid. The killer toxin is added in an amount effective to deactivate the fungus.

### BRIEF DESCRIPTION OF THE DRAWINGS

The present disclosure will be more readily understood from a detailed description of some example embodiments taken in conjunction with the following figures:

FIG. 1 is a chart showing the change in specific gravity over time during fermentation in the presence of a diastatic yeast.

FIG. 2 is a schematic showing a method of a post-screening assay according to an embodiment.

FIGS. 3 and 4 are charts showing the change in specific gravity over time during fermentation in the presence of a diastatic yeast with and without the presence of a killer yeast toxin, respectively.

FIG. 5 depicts a system according to an embodiment before and after fermentation.

### DETAILED DESCRIPTION

Various non-limiting embodiments of the present disclosure will now be described to provide an overall understanding of the principles of the structure, function, and use of the systems and methods disclosed herein. One or more examples of these non-limiting embodiments are illustrated in the accompanying drawings. Those of ordinary skill in the art will understand that systems and methods specifically described herein and illustrated in the accompanying drawings are non-limiting embodiments. The features illustrated or described in connection with one non-limiting embodiment may be combined with the features of other non-limiting embodiments. Such modifications and variations are intended to be included within the scope of the present disclosure.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment, or “in an embodiment” in places throughout the specification are not necessarily all referring to the same embodiment. Furthermore, the particular features, structures or characteristics may be combined in any suitable manner in one or more embodiments.

Described herein are example embodiments of systems and methods for treating a fermentation product to control or eliminate undesired attenuation of diastatic yeast. Diastatic yeasts are sensitive to killer yeast toxins, such as K1, K2, or K28. In one example embodiment, a killer yeast toxin is added during the fermentation process to deactivate the diastatic yeast thus controlling the attenuation. When the killer yeast toxin is introduced, the Saccharomyces cerevisiae var. diastaticus is inactivated or stabilized and further attenuation is prevented. Other example embodiments may include the use of killer yeast toxins to control not only diastatic Saccharomyces yeasts but also other non-Saccharomyces fungi (e.g., Candida, Dekkera, Aspergillus) that are vulnerable to these killer yeast toxins. Disclosed herein are methods for controlling over-attenuation of beer or another fermented liquid from microorganisms such as STA+ yeasts (e.g., Saccharomyces, Dekkera, Brettanomyces).

The examples discussed herein are examples only and are provided to assist in the explanation of the systems and methods described herein. None of the features or components shown in the drawings or discussed below should be taken as mandatory for any specific implementation of any of these the systems or methods unless specifically designated as mandatory. For ease of reading and clarity, certain components, modules, or methods may be described solely in connection with a specific figure. Any failure to specifically describe a combination or sub-combination of components should not be understood as an indication that any combination or sub-combination is not possible. Also, for any methods described, it should be understood that unless otherwise specified or required by context, any explicit or implicit ordering of steps performed in the execution of a method does not imply that those steps must be performed in the order presented but instead may be performed in a different order or in parallel.

Example embodiments described herein can include introducing a killer yeast toxin directly or indirectly during a fermentation process. For example, in an embodiment, isolated killer yeast toxin may be introduced when the desired level of attenuation has been reached. The killer yeast toxin may be a Saccharomyces cerevisiae killer toxin. Examples of the killer yeast toxin include, without limitation, those shown in Table 1 below, and combinations thereof. In an embodiment, more than one toxin at a time may be added, which may increase the effectiveness of the treatment against a wider range of STA+ yeasts. This can be a combination of any of the killer yeast toxins. In some embodiments, the concentration of the toxins in the fermentation batch may be in a range of about 2×10³ to about 4.4×10⁷ units ml⁻¹.

TABLE 1

| | | | | |
|---|---|---|---|---|
| | | | Toxic | |
| | | Toxin | Gene | |
| Genetic | Yeast Produced | Gene | Size | Killer Toxin |
| Basis | Toxin | Name | (Kbp) | Name |

Cytoplasmically inherited encapsulated double-stranded
RNA (dsRNA) viruses

| | | | |
|---|---|---|---|
| Saccharomyces | M1 | 1.8 | K1 |
| cerevisiae | | | |
| | M2 | 1.5 | K2 |
| | M28 | 1.9 | K28 |
| Ustilago maydis | M1/M2 | 1.4 | PI |
| | M2 | 0.98 | P4 |
| | M2 | 1.2 | P6 |
| Hanseniaspora | M | | |
| uvarum | | | |
| Phaffia rhodozyma | M | | |
| Zygosaccharomyces | M | | |
| bailii | | | |

Linear double strand plasmids

| | | |
|---|---|---|
| Kluyveromyces | pGkL1 | 8.8 |
| lactis | | |
| | pGkL2 | 13.4 |
| Pichia acacia | pPac 1-1 | 13.6 |
| | pPac 1-2 | 6.8 |
| Pichia inositovara | pPin 1-1 | 18 |
| | pPin 1-3 | 10 |

Chromosomal genes

| | | | |
|---|---|---|---|
| Saccharomyces | KHR | 0.9 | KHR |
| cerevisiae | | | |
| | KHS | 2.1 | KHS |
| Pichia farinosa | SMK1 | 0.6 | KK1 |
| Pichia | | | PMKT |
| membranifaciens | | | |
| | | | PMKT2 |
| Williopsis mraki | HMK | | HM-1, K-500 |
| Williopsis saturnus | HSK | | HSK |
| Williopsis saturnus | | | Wmkt |
| var mrakii | | | |

In an embodiment where a killer yeast toxin is indirectly introduced, the killer yeast toxin source may be introduced at or prior to the time when the desired level of attenuation has been reached. Killer yeasts are a group of yeasts that carry a viral like particle (VLP) that contains a gene for producing a killer yeast toxin. The killer yeasts may include, without limitation, S. cerevisiae strains that produce toxins K1, K2, or K28 or any other killer yeast toxin. In some embodiments, the transfer of the genes from the viral like particles from the killer yeast to a non-killer yeast may occur through protoplast fusion, transformation by plasmid, or by any other means for the purpose of preventing diastatic infections. Example methods of such gene transfer are disclosed in Zimmermann M., Sipiczki M., Protoplast Fusion of Yeasts, Nonconventional Yeasts in Biotechnology. Springer, Berlin, Heidelberg (1996), the disclosure of which is incorporated by reference herein in its entirety.

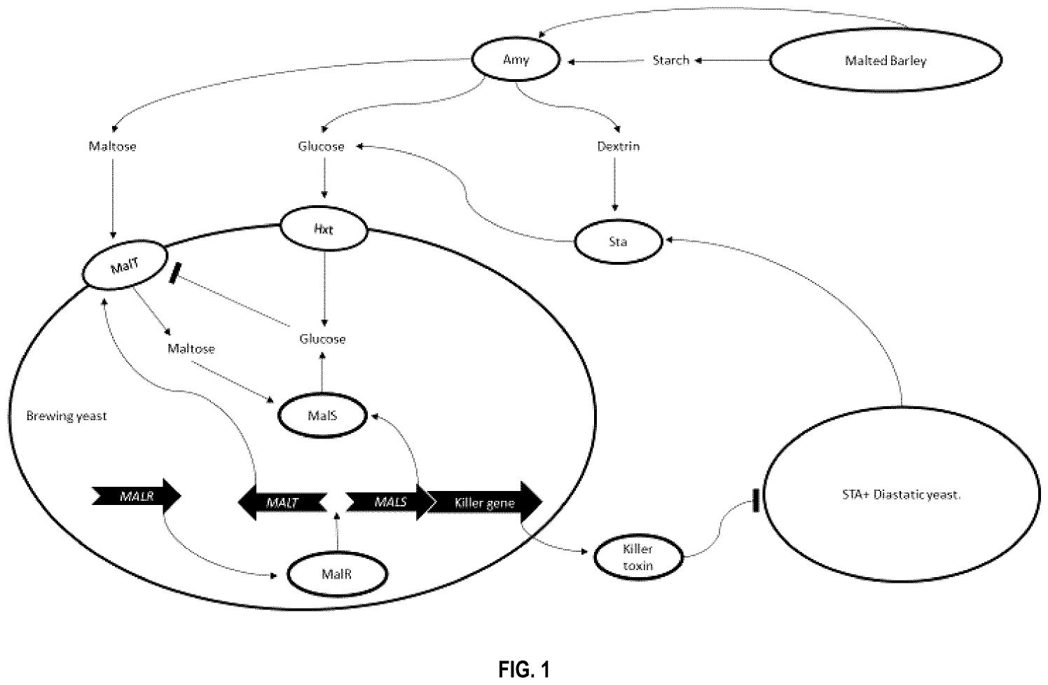

In another example embodiment, yeast capable of generating a killer yeast toxin is added during the fermentation process to deactivate the diastatic yeast. Various embodiments include a genetically modified host cell capable of producing a killer yeast toxin, where the genetically modified host cell is useful for producing a fermented liquid (e.g., beer). The host cell may be genetically edited, for example, using CRISPR editing to insert the genes into the chromosome of the non-killer yeast for the purpose of preventing diastatic infections. Example methods of such gene editing are disclosed in Ryan et al., CRISPR-Cas9 Genome Engineering in Saccharomyces cerevisiae Cells, Cold Spring Harb. Protoc (2016), the disclosure of which is incorporated herein in its entirety. In some embodiments, the host cell is a brewer's yeast. For example, the gene may be inserted into the maltose regulatory pathway (shown in FIG. 1). When the yeast is exposed to the brewing environment, which includes maltose, the production of the killer yeast toxin begins. An example embodiment provides a method of producing a fermented liquid, comprising: providing a host cell according to an embodiment and culturing said host cell in a suitable medium to produce the fermented liquid, such that the killer yeast toxin is produced to control the attenuation.

In various embodiments, killer yeast toxin may be produced and purified. The killer yeast toxin may then be used to attempt to rescue a fermentation from diastatic yeasts. In an embodiment, a broth of the killer yeast may be grown up and passed across a filter to remove living cells and concentrate the protein. The filtrate containing the protein as well as a number of other constituents may be substantially flavor neutral. The filtrate may also maintain the correct isoelectric point and pH for the specific killer protein being targeted. In some embodiments, the isoelectric point of pH in the fermentation batch after the addition of the toxin is in a range of 3.6 to 5.2 for the proteins to remain stable and in their natively folded confirmation. In some embodiments, yeast protein expression systems, such as those found in Saccharomyces cerevisiae and Picha pastoris, can be used to produce the protein.

In some embodiments, a method of treating a fermentation product to control or eliminate undesired attenuation of diastatic yeast may include a pre-treatment and/or post-treatment screening assay. The assays may act as an indicator whether treatment with the toxin is likely to have a positive outcome. For example, prior to using the killer yeast toxin for the purpose of clearing the product of any contaminating diastatic yeasts, the yeasts in the fermenter may be screened for the presence of genetic pathways that might confer resistance to the yeast to the killer yeast toxin. Example target genes for resistance screening to the K2 toxin include, without limitation: Beta glucan synthesis and assembly (THP1, KRE6, FKE1, SMI1, AIM26, TRS65, ANP1); N/O-linked protein glycosylation (OST3, PMT1, PMT2, ALG8, AMP1); Membrane organization and function (DRS2, LEM3, IGR1); and Cell cycle growth and proliferation (ELM1, CKA2, EGT2). Example target genes for sensitivity screening to the K2 toxin include, without limitation: General stress response/HOG pathway (HOG1, BEM2, BNI1, FUS3, RLM1); Cell wall biogenesis (LRG1, BEM2, BNI1, FUS3); and Phosoinositide synthesis (PLC1 and SAC1).

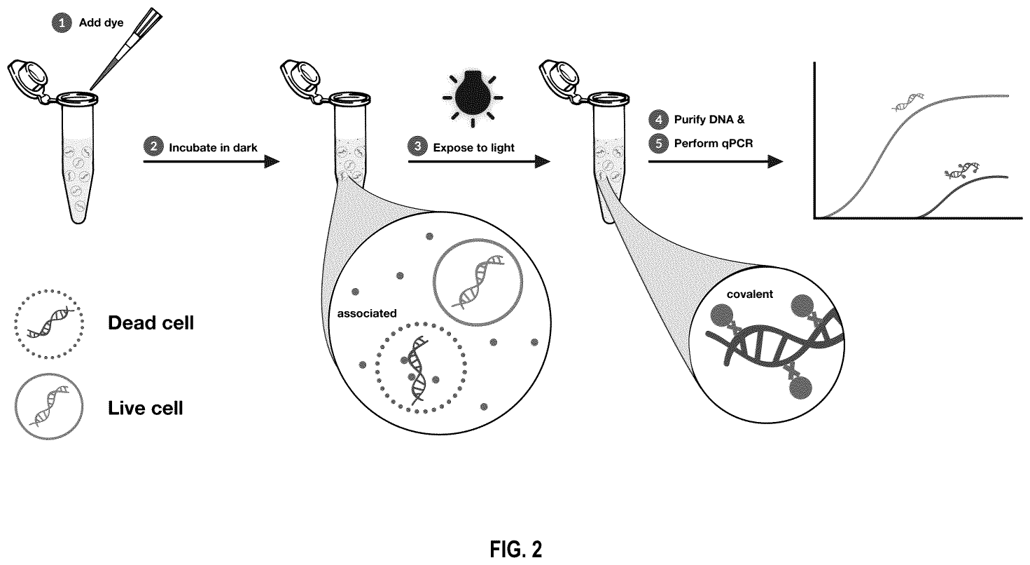

In an embodiment, after the killer yeast toxin is added, a post-treatment screen may include a viability PCR assay to ensure the product is clear of any remaining viable diastatic yeast cells. An understanding of the final community composition of the fermentation may be used to determine the efficacy of the treatment. Culture dependent methods such as cell counts lack the ability to distinguish between STA+ and STA-cells. Direct methods such as plate counts on media such as PFDM and LCSM time consuming and have been historically troubled by false positives. PCR primers for the STA family of genes will be used in conjunction with the PMAxx dye and a qPCR thermocycler to determine if any remaining viable STA1+ yeast cells are remaining as illustrated in FIG. 2. Example methods are disclosed in Emerson, J. B. et al., Schrödinger's microbes: Tools for distinguishing the living from the dead in microbial ecosystems, Microbiome, 5(1), 86(2017), the disclosure of which is incorporated by reference herein in its entirety.

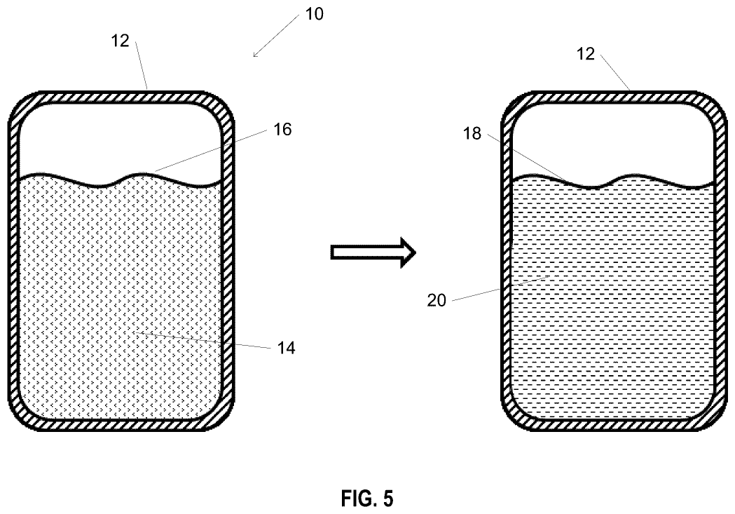

Example embodiments described herein can include a system for controlling attenuation in a fermentation process. For example, as shown in FIG. 5, the system 10 may include a fermentation system 12 configured to culture a fungus 14 in a medium 16 to produce a fermented liquid 18. The system 10 may include a killer toxin 20 configured to be added to the fermented liquid in an amount effective to deactivate the fungus 14.

The following examples are provided to help illustrate the present invention and are not comprehensive or limiting in any manner.

### Example 1

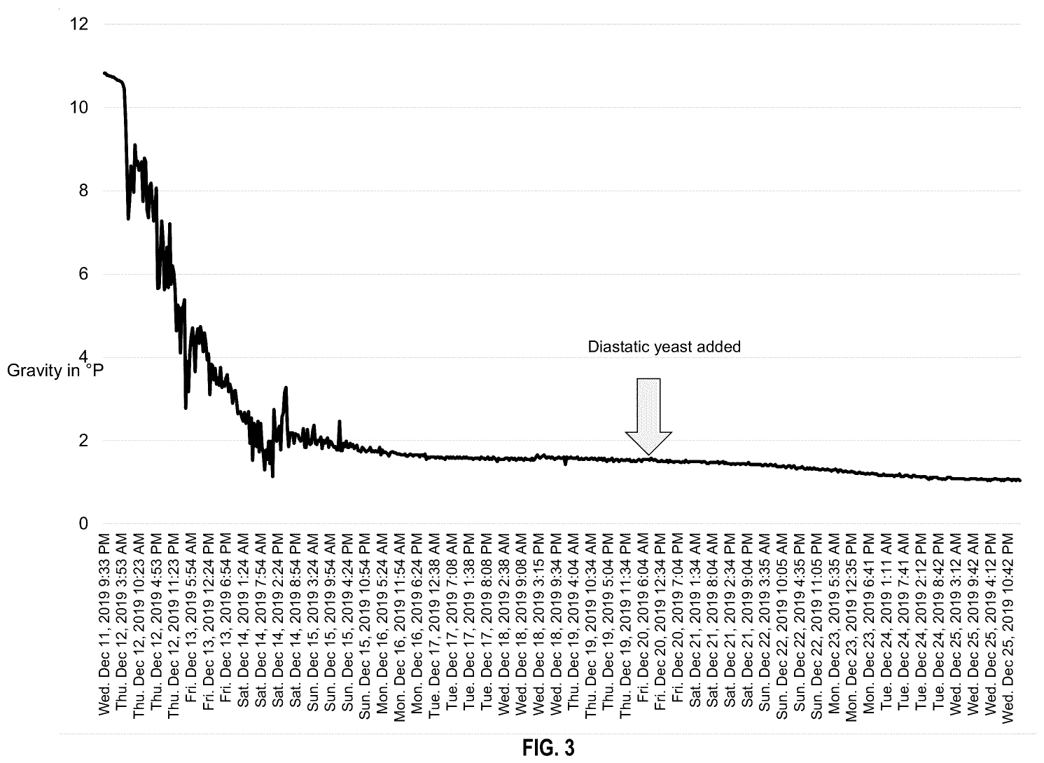

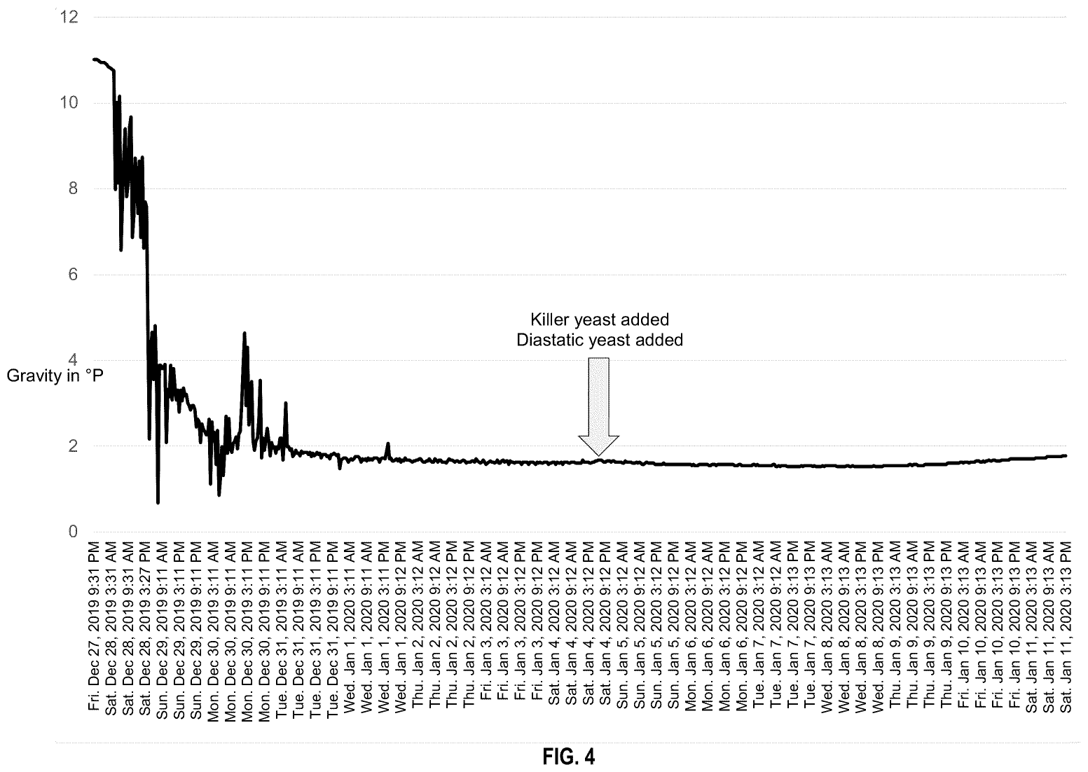

Two tests were completed in which 10,000 liters of beer were produced in each test. Using real time data collection via inline densitometer, samples were taken every 30 minutes with a total of 681 samples. As an indicator for attenuation, the specific gravity of the samples was recorded. FIGS. 3 and 4 show the specific gravity (degrees Plato) over time in Trial 1 and Trial 2, respectively.

When the specific gravity leveled off or became terminal, diastatic yeast was added. In Trial 2, killer yeast capable of producing the killer yeast toxin K2 was added at the same time as the diastatic yeast. As seen in FIG. 3, the specific gravity began dropping after the addition of the diastatic yeast in Trial 1. In contrast, as seen in FIG. 4, the specific gravity remained relatively level in Trial 2 indicating that the diastatic yeast was deactivated. There is a slight increase in the specific gravity in FIG. 4, which was attributed to the escape of ethanol while the diastatic yeast and killer yeast were added. The results show that superattuation was eliminated when the diastatic yeast was exposed to the killer yeast toxin K2 during the fermentation process.

The foregoing description of embodiments and examples has been presented for purposes of illustration and description. It is not intended to be exhaustive or limiting to the forms described. Numerous modifications are possible in light of the above teachings. Some of those modifications have been discussed, and others will be understood by those skilled in the art. The embodiments were chosen and described in order to best illustrate principles of various embodiments as are suited to particular uses contemplated. The scope is, of course, not limited to the examples set forth herein, but can be employed in any number of applications and equivalent devices by those of ordinary skill in the art. Rather it is hereby intended the scope of the invention to be defined by the claims appended hereto.

What is claimed is:

1. A method of controlling attenuation in a fermentation process comprising:
conducting a first screening assay on a beer;
adding a killer toxin to the beer after conducting the first screening assay; and
conducting a second screening assay on the beer after adding the killer toxin.

2. The method of claim 1, wherein adding the killer toxin comprises adding the killer toxin when a desired level of attenuation has been reached.

3. The method of claim 1, wherein adding the killer toxin comprises adding more than one type of killer toxin.

4. The method of claim 1, wherein adding the killer toxin comprises directly adding the killer toxin to the beer.

5. The method of claim 4, the method further comprising, before the killer toxin is added:
producing a broth by a killer yeast capable of generating the killer toxin; and
filtering the broth to provide the killer toxin.

6. The method of claim 1, wherein adding the killer toxin comprises indirectly adding the killer toxin to the beer.

7. The method of claim 6, wherein indirectly adding the killer toxin comprises adding viral like particles (VLPs) that contain a gene for producing the killer toxin.

8. The method of claim 7, wherein adding the VLPs comprises adding a killer yeast to the fermented liquid, wherein the killer yeast carries the VLPs.

9. The method of claim 6, wherein indirectly adding the killer toxin comprises adding a killer yeast capable of generating the killer toxin.

10. The method of claim 1, further comprising culturing a fungus in a medium to produce the beer, wherein the fungus is at least one of Saccharomyces, Dekkera, Brettanomyce, Candida, or Aspergillus.

11. The method of claim 10, wherein the killer toxin is added in an amount effective to deactivate the fungus.

12. The method of claim 10, wherein the first screening assay detects whether the fungus includes a genetic pathway that confers resistance to the killer toxin.

13. The method of claim 10, wherein the second screening assay detects a presence of remaining viable cells of the fungus.

14. The method of claim 1, wherein the killer toxin is at least one of K1, K2, or K28.
